(19) United States
(12) Patent Application Publication
(10) Pub. No.: US 2005/0251348 A1
(43) Pub. Date: Nov. 10, 2005

(54) Gene-to-gene graphical representation

(76) Inventors: Meng Fu Shih, Carlsbad, CA (US); May Fen Shih, Carlsbad, CA (US)

Correspondence Address: Meng Fu Shih, 6909 Catamaran Dr., Carlsbad, CA, 92009, US

(21) Appl. No.: 11/111,577

(22) Filed: Apr. 21, 2005

Related U.S. Application Data
(60) Provisional application No. 60/569,417, filed on May 7, 2004.

(51) Int. Cl. C12Q 1/68; G06F 19/00; G01N 33/48; G01N 33/50

(52) U.S. Cl. 702/20

(57) ABSTRACT

The present invention discloses a simple gene-to-gene graphical representation enabling quick, easy, and a bird's-eye view of a specific gene of interest and its relation to and from other genes that directly activate or inhibit this specific gene.

### CROSS-REFERENCE TO RELATED APPLICATIONS

This application claims priority benefits of provisional application Ser. No. 60/569,417, filed May 7, 2004, entire contents of which are incorporated herein by reference.

### FIELD OF THE INVENTION

The present invention generally relates to gene pathways, and more particularly, the present invention relates to a simple gene-to-gene graphical representation enabling quick, easy, and a bird's-eye view of a specific gene of interest and its relation to and from other genes that directly activate or inhibit this specific gene.

### BACKGROUND OF THE INVENTION

With the recent accumulation of genome sequences for many organisms, researchers now turn their attention to the identification and functions of proteins encoded by these genomes.

The present invention aims to create an online gene dictionary with graphical representation. The dictionary is designed to be user-friendly for those searching for a specific gene and/or browsing for interactions among genes. Many database websites provide lengthy and detailed papers on gene related information. However, it is time consuming for readers to go through such a huge amount of papers for information needed. The present invention on the other hand offers a quick and easy way for scientists to find needed information at a click on a computer. Through straight forward graphical representations, the present invention offers the users a condensed story about a specific gene and its interactions with other genes. Time-saving is therefore an advantage of the present invention, which is indeed an online dictionary about genes.

### SUMMARY OF THE INVENTION

In general, it is an object of the present invention to provide a graphical representation that shows a specific gene of interest and its relation to and from first tier genes that directly activate or inhibit this specific gene.

Some aspects of the invention relate to a gene-to-gene graphical representation comprising: a specific gene of interest; and at least one incoming first tier gene, wherein the specific gene is related to and derived from the at least one incoming first tier gene, the at least one incoming first tier gene directly activates or inhibits the specific gene.

In a further embodiment, the gene-to-gene graphical representation further comprises at least one outgoing first tier gene, wherein the at least one outgoing first tier gene is related to and derived from the specific gene, the at least one outgoing first tier gene being directly activated or inhibited by the specific gene.

In a further embodiment, the gene-to-gene graphical representation further comprises at least one incoming second tier gene, wherein the at least one incoming first tier gene is related to and derived from the at least one incoming second tier gene, the at least one incoming second tier genes activates or inhibits the at least one incoming first tier gene.

In a further embodiment, the gene-to-gene graphical representation further comprises at least one outgoing second tier gene, wherein the at least one outgoing second tier gene is related to and derived from the at least one outgoing first tier gene, the at least one outgoing second tier gene being directly activated or inhibited by the at least one first tier gene.

In a further embodiment, the gene-to-gene representation is displayed on a screen of a device selected from a group consisting of a computer monitor, a cellular phone, a smart phone, a personal digital assistant, a television, a handheld device, and a watch.

In one embodiment, the gene-to-gene representation further comprises a first icon for the specific gene, a second icon for the at least one incoming first tier gene to the specific gene, and a third icon for the at least one outgoing first tier gene from the specific gene. In another embodiment, the first, second and third icons of the gene-to-gene graphical representation are displayed on the screen with differentiable visual characteristic, wherein the visual characteristic is selected from a group consisting of font size, font color, font shape, font underline, font type, and combination thereof.

In one embodiment, the gene-to-gene graphical representation as a first site is linked to a second site via a transmitting means for linking information between the first and second sites, wherein the linking is carried out by a one-click action on any icon in the graphical representation that is displayed on the screen.

In one embodiment, the specific gene of the gene-to-gene graphical representation comprises certain basic description of the specific gene.

In one embodiment, the at least one incoming first tier gene of the gene-to-gene graphical representation comprises certain basic description of the at least one incoming first tier gene.

In one embodiment, the gene-to-gene graphical representation is shown as a hard copy selected from a group consisting of a flyer, a poster, a card, a sheet, and a print matter. In another embodiment, the graphical representation as a first site is linked to a second site via a transmitting means for linking information between the first and second sites. In still another embodiment, the transmitting means is selected from a group consisting of an internet, a wireless internet, a mobile internet, an intranet, a wireless intranet, a mobile intranet, an extranet, a mobile extranet, a fixed-line communication network, and a mobile communication network.

Some aspects of the invention relate to a method of quick searching a specific gene of interest, comprising: providing a gene-to-gene graphical representation comprising: a specific gene of interest with a first icon; at least one incoming first tier gene with a second icon, wherein the specific gene is related to and derived from the at least one incoming first tier gene, the at least one incoming first tier gene directly activates or inhibits the specific gene; and at least one outgoing first tier gene with a third icon, wherein the at least one outgoing first tier gene is related to and derived from the specific gene, the at least one outgoing first tier gene being directly activated or inhibited by the specific gene; and clicking on any of the icons to enable linking and displaying the graphical representation on a screen.

Some aspects of the invention relate to a method of quick searching a specific gene of interest, comprising: providing a gene-to-gene graphical representation comprising: a specific gene of interest with a first icon; at least one incoming first tier gene with a second icon, wherein the specific gene is related to and derived from the at least one incoming first tier gene, the at least one incoming first tier gene directly activates or inhibits the specific gene; at least one outgoing first tier gene with a third icon, wherein the at least one outgoing first tier gene is related to and derived from the specific gene, the at least one outgoing first tier gene being directly activated or inhibited by the specific gene at least one incoming second tier gene, wherein the at least one incoming first tier gene is related to and derived from the at least one incoming second tier gene, the at least one incoming second tier genes activates or inhibits the at least one incoming first tier gene, and at least one outgoing second tier gene, wherein the at least one outgoing second tier gene is related to and derived from the at least one outgoing first tier gene, the at least one outgoing second tier gene being directly activated or inhibited by the at least one outgoing first tier gene; and clicking on any of the icons to enable linking and displaying the graphical representation on a screen.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a conventional gene pathway that shows multiple tiers of genes with respect to a specific gene of interest.

FIG. 2 is one embodiment of a simple gene-to-gene graphical representation that shows a specific gene of interest and its relation to and from other genes that directly activate or inhibit this specific gene.

FIG. 3 is another embodiment of a simple gene-to-gene graphical representation that shows a specific gene of interest and its relation to and from other genes that directly activate or inhibit this specific gene.

FIG. 4 shows a graphical representation with an icon of the specific gene, icons of the incoming first tier genes to the specific genes, and icons of the outgoing first tier genes from the specific gene displayed with differentiable visual characteristics.

### DETAILED DESCRIPTION OF EXEMPLARY EMBODIMENTS

The following detailed description is of the best presently contemplated mode of carrying out the invention. This description is not to be taken in a limited sense, but is made merely for the purpose of illustrating general principles of embodiments of the invention.

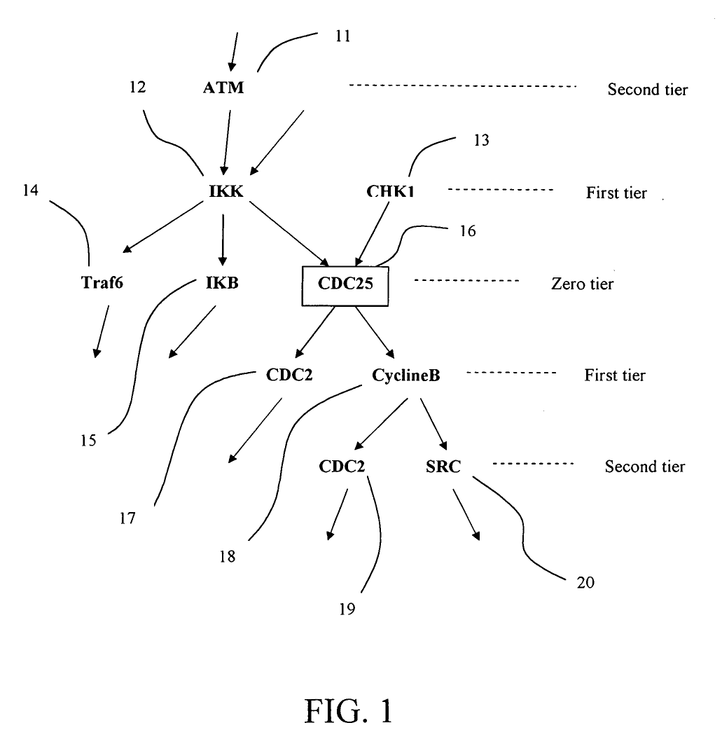

FIG. 1 shows a conventional, prior art gene pathway that displays multiple tiers of genes with respect to a specific gene (16) of interest. In some instances, the gene pathway may include tens or hundreds of relevant genes. By way of examples, the specific gene, CDC25 gene (16), consists of at least two incoming genes, CHK1 gene (13) and IKK gene (12), which can activate or inhibit the specific gene (16). Furthermore, the CDC25 gene consists of at least two outgoing genes, CDC2 gene (17) and CyclineB gene (18), which are activated or inhibited by the specific gene. Both the incoming genes and the outgoing genes that are directly involved in activating or inhibiting the specific gene are classified as the first tier genes, whereas the gene of interest is classified as the zero tier gene. Nevertheless, the prior art gene pathway further consists second tier genes, such as ATM gene (11), CDC2 gene (19), SRC gene (20) and higher tier genes. Such a gene pathway makes the graphical representation complicated and difficult to get a quick bird's-eye overview. Similarly, the zero tier gene, such as Traf6 gene (14) or IKB gene (15), can have its own incoming genes and the outgoing genes that are directly involved in activating and/or inhibiting the zero tier gene.

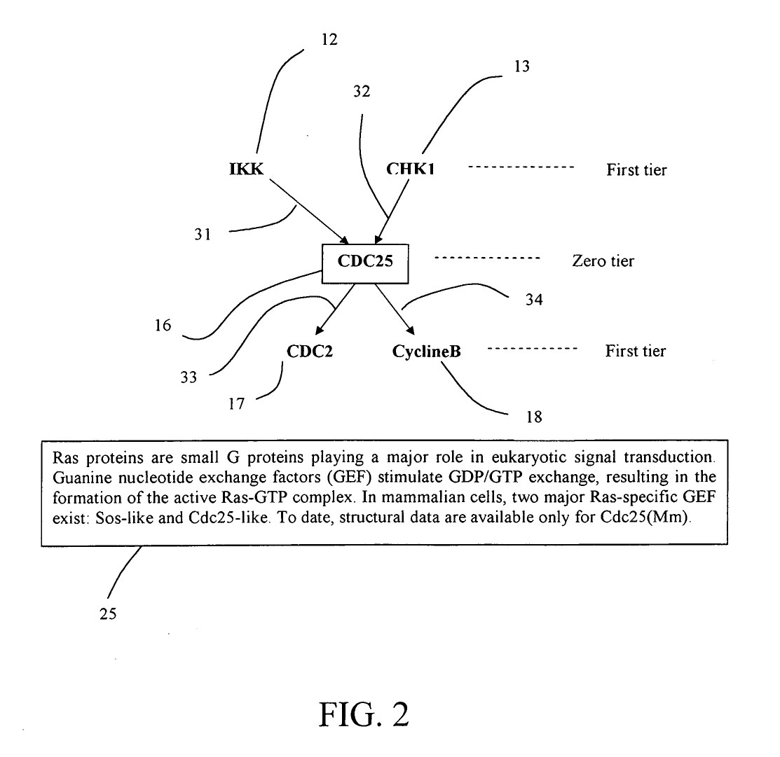

FIG. 2 shows a graphical representation that shows a specific gene CDC25 (16) of interest and its relation to its outgoing first tier genes, CDC2 gene (17) or CyclineB gene (18), and from its incoming first tier genes, IKK gene (12) or CHK1 gene (13) that directly activate or inhibit this specific gene. The first arrow (31) from IKK gene (12) and the second arrow (32) from CHK1 gene (13) indicate the incoming first tier genes that directly activate or inhibit the specific gene (16) of interest. Similarly, the third arrow (33) to CDC2 gene (17) and the fourth arrow (34) to CyclineB gene (18) indicate the outgoing first tier genes that are directly activated or inhibited by the specific gene (16) of interest.

Some aspects of the invention relate to a graphical representation that shows a specific gene of interest and its relation to the incoming first tier genes that directly activate or inhibit this specific gene. In a further embodiment, the graphical representation comprises certain basic description (25) of the specific gene. Similarly, the graphical representation could further comprise certain basic description of any of the incoming first tier genes that directly activate or inhibit the specific gene and/or certain basic description of any of the outgoing first tier genes that are directly activated or inhibited by the specific gene.

In a further embodiment, the graphical representation may be displayed on a screen of a device selected from a group consisting of a computer monitor, a cellular phone, a smart phone, a personal digital assistant, a television, a handheld device, a watch, and the like. In still a further embodiment, the graphical representation may be shown as a hard copy selected from a group consisting of a flyer, a poster, a card, a sheet, a print matter, and the like.

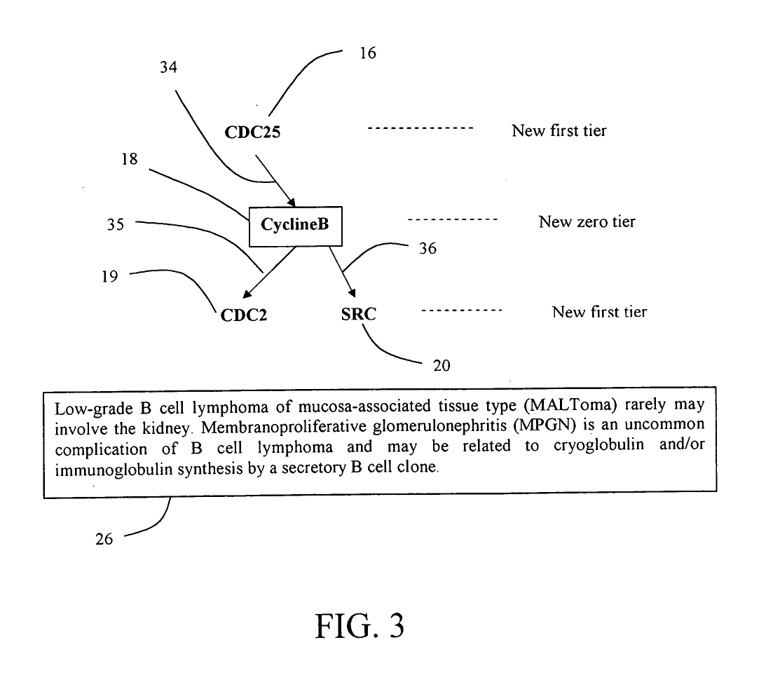

FIG. 3 shows another embodiment of a simple gene-to-gene graphical representation that shows a second specific gene, CyclineB gene (18) of interest as the new zero tier gene and its relation to new outgoing first tier genes, CDC2 gene (19) and SRC gene (20), and from new incoming first tier genes, such as CDC25 gene (16), wherein those incoming and outgoing first tier genes directly activate/inhibit this specific gene or are directly activated/inhibited by this second specific gene (18) referred herein as the new zero tier gene. By way of examples in FIG. 3, the first arrow (34) from CDC25 gene (16) indicates the incoming first tier gene that directly activates or inhibits the specific gene (18) of interest. Similarly, the second arrow (35) to CDC2 gene (19) and the third arrow (36) to SRC gene (20) indicate the outgoing first tier genes that are directly activated or inhibited by the specific gene (18) of interest. In some embodiment, the graphical representation comprises certain basic description (26) of the specific gene (18).

The second specific gene (18) is linked to the first specific gene (16). In a preferred embodiment, the graphical representation is linked to another site via a transmitting means for linking information between two or more linkable sites (for example, CDC25 gene site is linked to CHK1 gene site, IKK gene site, CDC2 gene site, and CyclineB gene site), wherein the transmitting means comprises any of the following: an internet, a wireless internet, a mobile internet, an intranet, a wireless intranet, a mobile intranet, an extranet, a mobile extranet, a fixed-line communication network, a mobile communication network, and the like.

In a further embodiment, the linking is carried out by a one-click action on the icon of any gene in the graphical representation that is displayed on the screen.

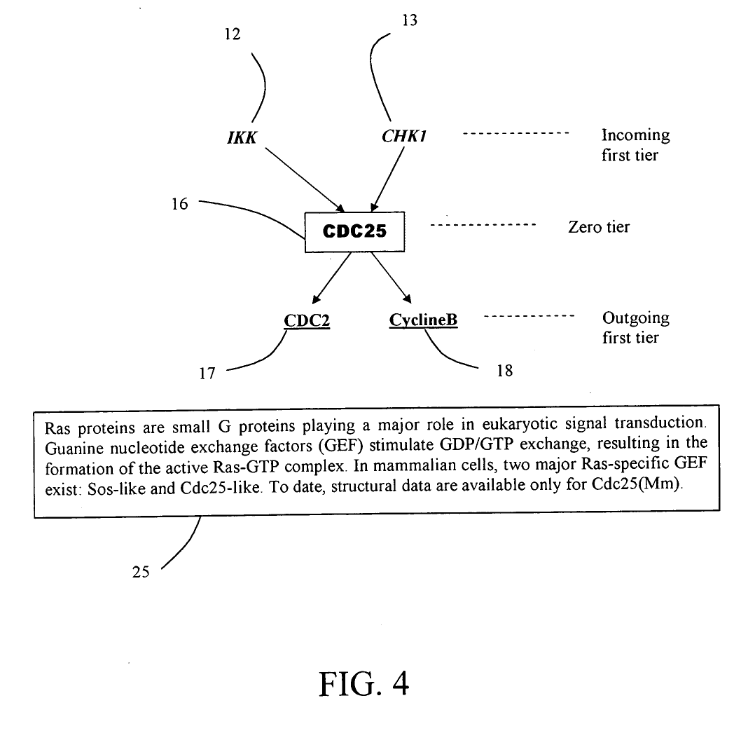

FIG. 4 shows a graphical representation with an icon of the specific gene, icons of the incoming first tier genes to the specific genes, and icons of the outgoing first tier genes from the specific gene displayed with differentiable visual characteristics, wherein the visual characteristics is selected from a group consisting of font size, font color, font shape, font underline, font type, combination thereof, and the like. By way of examples, the icon of the specific gene (16) is with an Arial Black font type, the icons of the incoming first tier genes are with italic font type, and the icons of the outgoing first tier genes are with underline characteristics.

In general, it is an object of the present invention to provide a graphical representation that shows a specific gene of interest and its relation to incoming first tier genes that directly activate or inhibit this specific gene. In a further embodiment, the graphical representation may comprise second tier genes that are directly related to and from the first tier genes, wherein the second tier genes (so named as reference to the zero tier gene) directly activate/inhibit one of the first tier genes or are directly activated/inhibited by one of the first tier genes. In general, the gene-to-gene (gene2gene™) graphical representation of the present invention is limited to no more than the second tier genes that enable quick, easy, and a bird's-eye view of a specific gene of interest and its relationship to the incoming and outgoing genes that involved in activating or inhibiting this specific gene.

Some aspects of the invention relate to a method for quick searching a specific gene of interest, comprising: providing a graphical representation showing the specific gene and its relation to either incoming or outgoing first tier genes that directly associate with activating or inhibiting this specific gene or being activated or being inhibited by this specific gene; and clicking on an icon of the specific gene to enable linking and displaying the graphical representation on a displaying device. In a further embodiment, the graphical representation of the method further comprises second tier genes (both incoming and outgoing) that are directly related to and from the respective first tier genes, wherein the second tier genes directly activate/inhibit one of the first tier genes or are directly activated/inhibited by one of the first tier genes.

From the foregoing descriptions, it should now be appreciated that a novel graphical representation and methods for promoting bioinformatics has been disclosed for genetics and proteomics applications. While the invention has been described with reference to a specific embodiment, the description is illustrative of the invention and is not to be construed as limiting the invention. Various modifications and applications may occur to those who are skilled in the art, without departing from the true spirit and scope of the invention.

1. A gene-to-gene graphical representation comprising:
a specific gene of interest; and
at least one incoming first tier gene, wherein said specific gene is related to and derived from said at least one incoming first tier gene, said at least one incoming first tier gene directly activates or inhibits the specific gene.

2. The graphical representation of claim 1, further comprising at least one outgoing first tier gene, wherein said at least one outgoing first tier gene is related to and derived from said specific gene, said at least one outgoing first tier gene being directly activated or inhibited by the specific gene.

3. The graphical representation of claim 2, further comprising at least one incoming second tier gene, wherein said at least one incoming first tier gene is related to and derived from the at least one incoming second tier gene, said at least one incoming second tier genes activates or inhibits the at least one incoming first tier gene.

4. The graphical representation of claim 3, further comprising at least one outgoing second tier gene, wherein said at least one outgoing second tier gene is related to and derived from said at least one outgoing first tier gene, said at least one outgoing second tier gene being directly activated or inhibited by the at least one first tier gene.

5. The graphical representation of claim 2, wherein the representation is displayed on a screen of a device selected from a group consisting of a computer monitor, a cellular phone, a smart phone, a personal digital assistant, a television, a handheld device, and a watch.

6. The graphical representation of claim 5, further comprising a first icon for the specific gene, a second icon for the at least one incoming first tier gene to the specific gene, and a third icon for the at least one outgoing first tier gene from the specific gene.

7. The graphical representation of claim 6, wherein the first, second and third icons are displayed on the screen with differentiable visual characteristic, wherein said visual characteristic is selected from a group consisting of font size, font color, font shape, font underline, font type, and combination thereof.

8. The graphical representation of claim 7, wherein the graphical representation as a first site is linked to a second site via a transmitting means for linking information between the first and second sites, wherein the linking is carried out by a one-click action on any icon in the graphical representation that is displayed on the screen.

9. The graphical representation of claim 1, wherein said specific gene comprises certain basic description of the specific gene.

10. The graphical representation of claim 1, wherein said at least one incoming first tier gene comprises certain basic description of said at least one incoming first tier gene.

11. The graphical representation of claim 1, wherein the graphical representation is shown as a hard copy selected from a group consisting of a flyer, a poster, a card, a sheet, and a print matter.

12. The graphical representation of claim 1, wherein the graphical representation as a first site is linked to a second site via a transmitting means for linking information between the first and second sites.

13. The graphical representation of claim 12, wherein the transmitting means is selected from a group consisting of an internet, a wireless internet, a mobile internet, an intranet, a wireless intranet, a mobile intranet, an extranet, a mobile extranet, a fixed-line communication network, and a mobile communication network.

14. A method of quick searching a specific gene of interest, comprising:
providing a gene-to-gene graphical representation comprising: a specific gene of interest with a first icon; at least one incoming first tier gene with a second icon, wherein said specific gene is related to and derived from said at least one incoming first tier gene, said at least one incoming first tier gene directly activates or inhibits the specific gene; and at least one outgoing first tier gene with a third icon, wherein said at least one outgoing first tier gene is related to and derived from said specific gene, said at least one outgoing first tier gene being directly activated or inhibited by the specific gene; and
clicking on any of the icons to enable linking and displaying the graphical representation on a screen.

15. A method of quick searching a specific gene of interest, comprising:
providing a gene-to-gene graphical representation comprising: a specific gene of interest with a first icon; at least one incoming first tier gene with a second icon, wherein said specific gene is related to and derived from said at least one incoming first tier gene, said at least one incoming first tier gene directly activates or inhibits the specific gene; at least one outgoing first tier gene with a third icon, wherein said at least one outgoing first tier gene is related to and derived from said specific gene, said at least one outgoing first tier gene being directly activated or inhibited by the specific gene at least one incoming second tier gene, wherein said at least one incoming first tier gene is related to and derived from the at least one incoming second tier gene, said at least one incoming second tier genes activates or inhibits the at least one incoming first tier gene, and at least one outgoing second tier gene, wherein said at least one outgoing second tier gene is related to and derived from said at least one outgoing first tier gene, said at least one outgoing second tier gene being directly activated or inhibited by the at least one outgoing first tier gene; and
clicking on any of the icons to enable linking and displaying the graphical representation on a screen.
